(19) United States
(12) Patent Application Publication
(10) Pub. No.: US 2026/0114755 A1
(43) Pub. Date: Apr. 30, 2026

(54) IMAGE CYTOMETRY-BASED NON-INVASIVE WHITE BLOOD CELL COUNT FROM FINGERTIP VIDEOS

(71) Applicant: Marquette University, Milwaukee, WI (US)

(72) Inventors: Iqbal Ahamed Sheikh, Milwaukee, WI (US); Nafi US Sabbir Sabith, Milwaukee, WI (US)

(73) Assignee: Marquette University, Milwaukee, WI (US)

(21) Appl. No.: 19/473,737

(22) PCT Filed: Apr. 10, 2024
(86) PCT No.: PCT/US2024/023873

§ 371 Date: Oct. 8, 2025

Related U.S. Application Data
(60) Provisional application No. 63/495,417, filed on Apr. 11, 2023.

(51) Int. Cl. A61B 5/145; A61B 5/00; A61B 5/024; A61B 5/029; G06V 10/14; G06V 10/762; G06V 40/10

(52) U.S. Cl. CPC A61B 5/145; A61B 5/0077; A61B 5/02416; A61B 5/029; A61B 5/742; G06V 10/14; G06V 10/762; G06V 40/11; A61B 2562/0223; G06V 2201/03

(57) ABSTRACT

Systems and methods for non-invasive measurement of white blood cell (WBC) count acquire a plurality of images with a camera. A light source illuminates the target while a magnetic applies a magnetic field to the target. A processor receives the plurality of images. The processor is configured to extract at least one image attribute from each image of the plurality of images and apply a K-means clustering technique to the extracted image attributes to classify the plurality of images. The processor is configured to calculate a WBC rate from the classified images and use a cardiac output with the WBC rate to calculate the WBC count.

### CROSS-REFERENCE TO RELATED APPLICATIONS

The present application claims priority of U.S. Provisional Patent Application No. 63/495,417, filed on Apr. 11, 2023, the contents of which are incorporated by reference in their entirety.

### BACKGROUND

Blood composition is important for diagnostic purposes. Laboratory tested blood samples remain the gold standard for blood composition determinations. Other areas of circulatory monitoring can be done non-invasively, for example, pulse monitoring and SP 02. More recently, non-invasive techniques have been developed for blood composition purposes. US patent application publication number 2021/0007648 entitled “method and apparatus for non-invasive hemoglobin level prediction”, which is incorporated by reference herein in its entirety, discloses an image-based hemoglobin estimation tool for measuring hemoglobin from video data of an illuminated finger. The method includes acquiring a time-based series of images of the finger ventral pad-tip illuminated from the dorsal side of the finger with a near-infrared light responsive to blood hemoglobin, and white light, and acquiring a second time-based series of images of the finger ventral pad-tip illuminated from the dorsal side of the finger with a near-infrared light responsive to blood plasma, and white light. Each image in each of the first and second time-based series is divided into groups of blocks. A time series signal is generated from each block, and at least one Photoplethysmography (PPG) cycle is identified from each of the time series signals, including a systolic peak and a diastolic peak. The PPG cycles are processed to determine blood hemoglobin levels.

While hemoglobin levels are an important aspect of blood composition, the inventors have sought to develop a non-invasive test and device for performing the test, to determine white blood cell count. White blood cells (WBC) are important parts of our immune system and protect against infections by eliminating viruses, bacteria, parasites, and fungi. There are five types of WBC. These are called lymphocytes, monocytes, eosinophils, basophils, and neutrophils. The number of WBC types and the total number of WBCs provide important information about health status. Diseases such as leukemia, AIDS, autoimmune diseases, immune deficiencies, and blood diseases can be diagnosed with reference to the number and types of WBCs. Different immune symptoms can change the percentages and the number distributions of various WBCs. Bacterial infections can exhibit increased Neutrophil count while viral infections raise lymphocyte counts. As noted above, the most common way of determining white blood cell levels is the Complete Blood Count (CBC) lab test. This often remains inaccessible, expensive, and time-consuming.

### BRIEF DISCLOSURE

Examples of systems for non-invasive measurement of white blood cell (WBC) count include a camera configured to acquire a plurality of images, a light source, and a magnet positioned relative to the camera. A processor is configured to receive the plurality of images. The processor is configured to extract at least one image attribute from each image of the plurality of images and apply a K-means clustering technique to the extracted image attributes to classify the plurality of images. The processor is configured to calculate a WBC rate from the classified images and use a cardiac output with the WBC rate to calculate the WBC count.

In additional examples of systems for non-invasive measurement of WBC count, the processor is configured to determine a WBC level from the WBC count and a graphical display is operable by the processor to visually present the WBC level on the graphical display. The light source may be configured to emit light about 430 nm wavelength. A polarizing band pass filter may be configured with a pass band centered on 430 nm wavelength. The system may be configured for a user to insert a finger along a first direction to position the finger relative to the camera and the magnet is positioned to produce a magnetic field generally parallel to the first direction. The image attributes extracted from each image in of the plurality of images may include a peak value of light absorbed in a red channel and a difference between two successive peak values of light absorbed in the red channel. The image attributes extracted from each image in the plurality of images may include a peak value of light absorbed in a blue channel and a difference between two successive peak values of light absorbed in the blue channel. A magnetometer may be positioned proximate to the camera and the processor is configured to measure a magnetic field proximate to the camera with the magnetometer and compare the measured magnetic field to a threshold value before receiving the plurality of images.

In further examples of the systems for non-invasive measurement of WBC count, the processor is further configured to calculate the cardiac output from the plurality of images. The extracted at leas one image attribute is a maximum pixel intensity value and the K-means clustering classifies the at least one extracted image attribute into a plurality of K-clusters. The WBC rate is calculated using a K-cluster of the plurality of K-clusters selected as having a lowest maximum pixel intensity threshold value. The maximum pixel intensity value is a maximum pixel intensity value in a red channel of each image. The processor is configured to calculate the WBC rate as a count of the images in the selected K-cluster divided by a count of the number of images classified. The plurality of K-clusters includes our clusters. A first cluster of the plurality of K-clusters has a highest maximum pixel intensity threshold value. A second cluster of the plurality of K-clusters has a second highest maximum pixel intensity threshold value. A third cluster of the plurality of K-clusters has a third highest maximum pixel intensity threshold value. A fourth cluster of the plurality of K-clusters has the lowest maximum pixel intensity threshold value, and the fourth cluster is the selected cluster.

A method of non-invasive measurement of white blood cell (WBC) count includes illuminating a target with light centered on a 430 nm wavelength. The target is subjected to a static magnetic field. A plurality of images of the target are acquired. Image attributes are extracted from each image of the plurality of images. The images of the plurality of images are classified using a K-means classifying algorithm to define classification boundaries to a plurality of K-clusters. A WBC rate is calculated from the classified images. The WBC count is calculated from the WBC rate and a cardiac output.

Additional examples of the method of non-invasive measurement of WBC count includes the WBC rate being calculated from red channel image data from the classified images. A cardiac output may be obtained from a lookup table based upon patient demographic information. A cardiac output may be calculated from the plurality of images of the target. This may include obtaining a photoplethysmography (PPG) signal from the plurality of images. An inflection point area (IPA) is calculated from the PPG signal. A normalized harmonic area (NHA) is obtained from the PPG signal. An inflection harmonic area ratio (IHAR) is calculated between the IPA and the NHA. The cardiac output is calculated from the IHAR. Clinical standards may be applied to the calculated WBC count to determine a WBC level. A magnetic field may be measured with a magnetometer positioned proximate the camera and a strength of the static magnetic field confirmed before acquiring the plurality of images of the target. At least one of a magnetic field, a light intensity, and a patient motion may be measured. The at least one of the measured magnetic field, light intensity, and patient motion is compared against at least one respective predetermined threshold before the plurality of images of the target are acquired.

### BRIEF DESCRIPTION OF THE DRAWINGS

FIG. 1 is an end view of an example system for measuring white blood cell count.

FIG. 2 is a system diagram of the system for measuring white blood cell count.

FIG. 3 is an example top view of the system for measuring white blood cell count.

FIG. 4 is a perspective view of the system for measuring white blood cell count.

FIG. 5 is a diagram of the magnetic effect on RBC flow.

FIG. 6 is a flow chart that depicts an example of a method of measuring white blood cell count.

FIG. 7 is a flow chart that depicts an example of a method of extracting features from image data.

FIG. 8 is an example table of features extracted from the images.

FIG. 9 is a graph presenting the MaxRed values for each image after classification.

FIG. 10 are comparative graphs of extracted features from image data collected with and without the effect of a magnetic field.

FIG. 11 are exemplary graphs of raw PPG and denoised PPG signals obtained from image data.

### DETAILED DISCLOSURE

Systems and methods for non-invasively measuring white blood cell (WBC) count are disclosed herein. The systems and methods combine an apparatus for the collection of video image data and image processing techniques to non-invasively measure WBC count.

While not necessary for all implementations of the presently disclosed system and method, one advantage of the disclosed system and method is the potential to be implemented by leveraging the camera and processor of a standard smartphone device. The leverage of this ubiquitous and comparatively inexpensive device greatly increases the potential patient access to WBC count information.

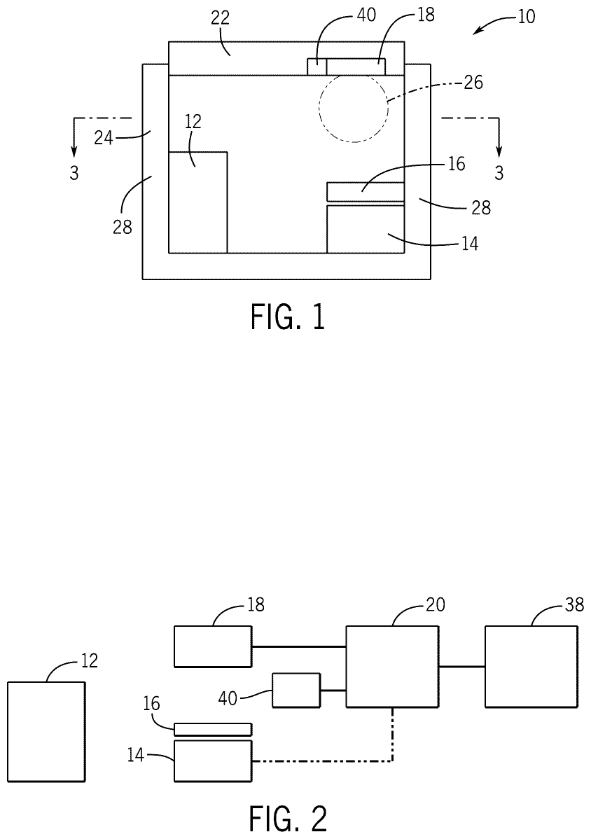

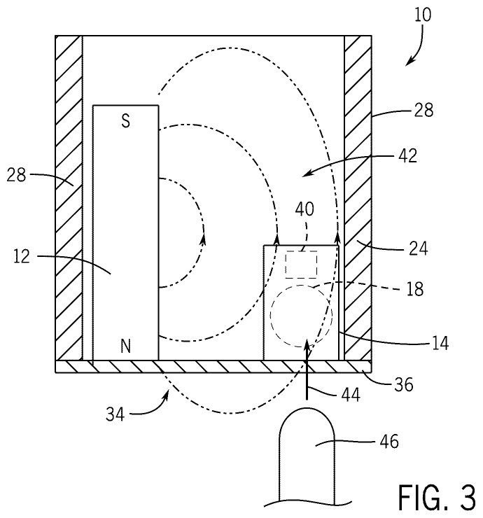

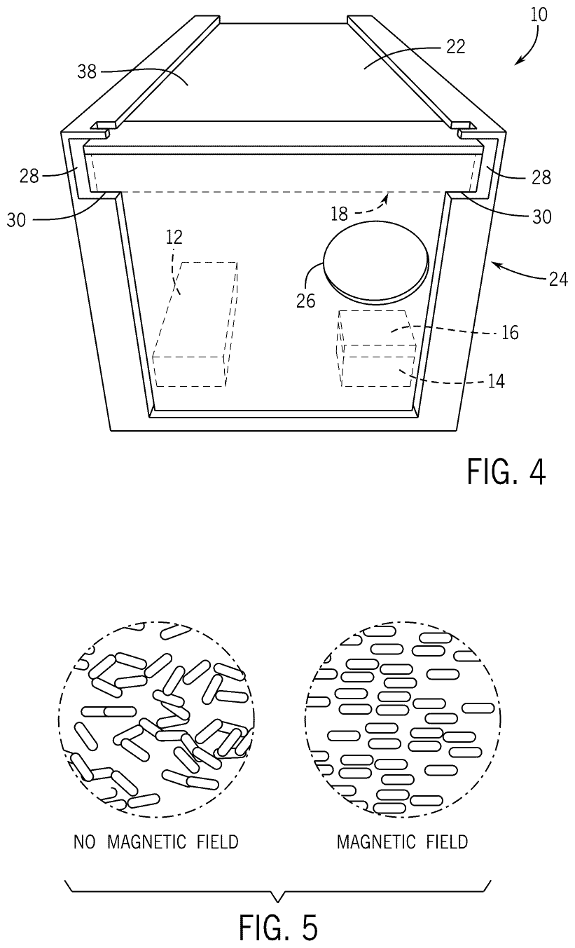

FIG. 1 is an end view of an example system 10 for measuring white blood cell count. FIG. 2 is a system diagram of the system 10 and FIG. 3 is an example top view of the system 10, taken along line 3-3 in FIG. 1. FIG. 4 is a perspective view of the system 10. The system 10 includes a magnet 12, a light source 14, a filter 16, and a camera 18. In examples, the camera 18, as well as a computer 20 for processing the acquired image data as described herein, may be provided by a smartphone 22. In examples as described herein, the system 10 further includes a magnetometer 40. The magnetometer 40 may be incorporated into the circuitry of the smartphone 22. The computer 20 receives inputs and in response to the inputs, produces outputs according to the methods and processes as described herein. As noted, in examples, the computer 20 may be provided as part of a smartphone 22. In other examples, the system 10 may be constructed as a stand-alone device, exemplarily carried out with a Raspberry Pi or other single board computer (SBC) which includes a microprocessor and associated computing components for e.g. power management, communication, and/or memory. The computer 20 executes computer readable code stored in a non-transient computer readable medium, causing the computer 20 to carry out the functions as described herein.

The system 10 may further include housing 24 configured to retain the described components therein including the smartphone 22 in intended relative positions. The housing 24 includes side walls 28 that further define a ledges 30 which support the smartphone 22 in a set position. Lips 32 above the ledges 30 retain the smartphone 22 in this position. The smartphone 22 is exemplarily slid into the housing 24 between the ledges 30 and the lips 32. A front end 34 of the housing 24 is exemplarily defined by a movable gate 36. The gate 36 is vertically slid into place between the side walls 28 and may further function to retain the smartphone 22 in the set position. The gate 36 of the housing 24 includes an aperture 26. The aperture 26 is located in the gate 36 relative to the set position of the smartphone 22 so as to facilitate and prompt proper positioning of a fingertip of a patient relative to the camera 18 for image acquisition as described herein. While not depicted, the housing 24 may include further internal alignment structures or a cradle to facilitate patient positioning of the finger relative to the magnet 12, light source 14, and camera 18. After the WBC count is determined according to the system and method as described herein, the computer 20 operates to visually present the results on a graphical display 38 which, too, may be a component of the smartphone 22.

Red blood cells (RBC) dominate the optical properties of flowing blood primarily due to the presence of hemoglobin. The presently disclosed system uses two systems to condition the environment in which the video image data is collected in a manner to enhance the distinction of the optical properties of WBCs from RBCs. Illumination of the fingertip with specified wavelength(s) of light increases the contrast between red blood cells and white blood cells. A magnetic field applied to the fingertip regulates the flow of red bloods cells during data collection.

UV wavelengths (e.g. 250 nm-370 nm) and the lower end of visible light wavelengths (e.g. 370-580) enhance fluorescence properties of WBC. For the systems disclosed herein wavelengths between 366-488 nm may be used, or more specifically wavelengths between 366-436, even more specifically a wavelength of 430 nm may be preferred for WBC fluorescence. In application, light about the 430 nm wavelength, for example +/−5 nm may be used. Illuminating the fingertip with light of a wavelength centered on 430 nm frequency maximized the contrast between the red blood cells and flow gaps. With this contrast maximized, the WBCs appear as optical absorption gaps in the bloodstream when illuminated. In still further examples, the fluorescence of different kinds of WBCs (as noted above) when taken alone may be enhanced at different wavelengths between 250-580 nm. In some cases between 250-370 nm, 370-440 nm, or 440-580 nm however, through experimentation, the inventors have found light of a wavelength of 430 nm enhanced WBC fluorescence and contrast with RBC. When imaged in isolation at this wavelength, the WBCs appear transparent and the RBCs dark, and in the resulting images the WBCs appear as optical absorption gaps in a continuous RBC stream.

In the system 10, the light source 14 provides the light at the wavelength(s) described herein. In an example, this may be from light source that provides a dedicated wavelength(s), for example a wavelength-specific LED or LEDs. In another example, as depicted in FIG. 1, a filter 16 may be combined with a source 14 of white light. In an example, the filter 16 may be a polarizing band pass filter with a pass band centered on a specified light wavelength, e.g. 430 nm with, for example, a 10 nm pass band.

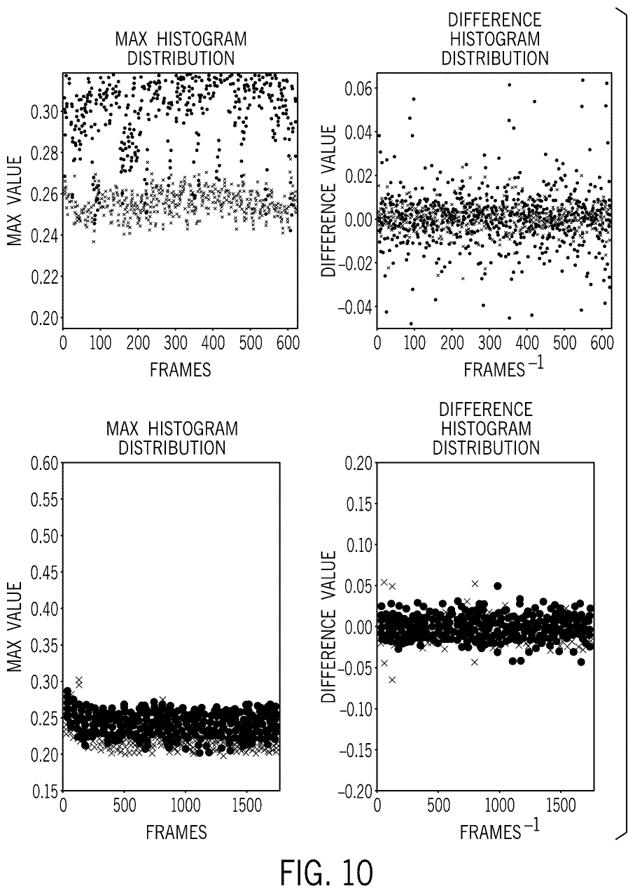

The system 10 further includes the magnet 12 arranged adjacent to the camera 18 and the light source 14. FIG. 3 better depicts the positioning of the magnet 12. The magnet 12 is oriented parallel to the target anatomy 46, for example a finger of the patient, when the finger is inserted in the direction of arrow 44 into the housing 24 through the aperture 26 between the light source 14 and the camera 18. The magnet 12 is a static magnet, which produces a field 42 between the North and South poles of the magnet. The orientation of the field 42 is thus in general parallel alignment with the general direction of blood flow (to and from the finger tip) within the target anatomy 46, for example, a finger. The field 42 is further in general parallel alignment with the direction 44 in which the target anatomy 46 is inserted into the housing 24. This curatively applied magnetic field near the magnetometer 40 can repulse the negative charge from hemoglobin in the target anatomy 46. This static magnetic field rotates the dominant RBC in the target anatomy 46 into alignment with the magnetic field and helps to regulate the flow of the RBC within the target anatomy 46. This makes the RBC flow more coherent, reducing RBC fluorescence noise which would otherwise overwhelm the WBC fluorescence. FIG. 5 depicts an example of RBC alignment and coherence when a magnetic field is generally aligned with the direction of blood flow. The magnetic field penetrates through the skin and into the surrounding tissue and bloodstream. The iron in the blood is attracted to and aligns with the magnetic field, this causes movement within the bloodstream and the increased activity and alignment improves blood flow. FIG. 10 presents comparative MaxRed/MaxBlue Histograms and RedDiff/BlueDiff Histograms for data obtained without and with the presence of the magnetic field.

As will be described herein, the camera 18 is used to acquire video image data, or that is a sequence of frames (e.g. images) acquired in rapid succession over a duration of time. In the example herein, the image data is acquired at a sampling rate of 30 images per second and captured for a duration of 60 seconds, for approximately 1800 images. It will be recognized that other sampling rates and other durations may be used while remaining within the scope of the present disclosure. In one aspect, the quality of the obtained image data may be initially evaluated with reference to an RGB motion sensor associated with the camera 18. The patient is instructed to insert a finger tip through the aperture 26 and to press the finger tip against the camera 18. The RGB motion sensor produces a signal indicative of motion relative to the sensor. Analysis of this signal by the computer 20 provides an evaluation if the image data contains motion artifacts. If the RGB motion sensor indicates motion of the finger tip, and thus motion artifacts in the image data, the computer 20 may operate the graphical display 38 to present an instruction to re-acquire the image data. With a set of image data acquired that is free of motion artifacts, the remaining motion in the acquired video image data is thus attributed to blood flow within the finger.

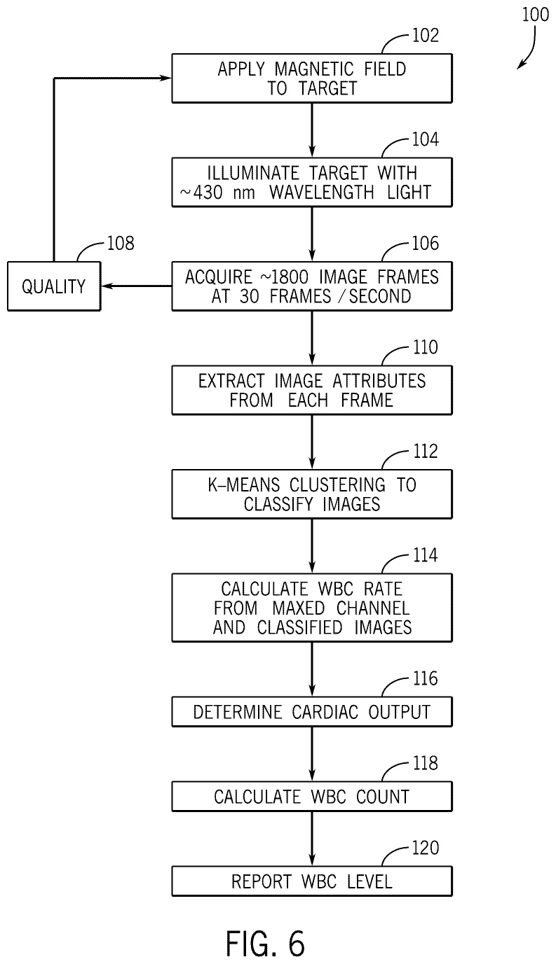

The above-noted features contribute to the collection of image data from which WBC count can be derived. WBC count is determined from a calculated WBC rate and a blood flow volume. The processing of the image data performed the computer to arrive at this output is detailed herein. FIG. 6 is a flow chart that depicts an example of a method 100 for non-invasively obtaining WBC count.

The patient inserts a finger into the system 10 and presses the finger against the camera 18. At 102, as described above, the magnetic field is applied to the target (finger). In an example, this is a static magnetic field. At 104, the target is illuminated with light, exemplarily light having the wavelength of 430 nm. It will be recognized that various ranges of about 430 mn may be used within the scope of embodiments as disclosed herein. The computer 20 may operate an initial routine to determine if the analyzed anatomy is properly positioned relative to the camera 18. In an example, the display 38 may be operated to give an indication of proper placement, before data recording begins. In an example, data recordation may being automatically once the finger has been in proper placement for a predefined amount of time, e.g. 0.5-2.0 seconds. At 106, the camera 18 operates to exemplarily acquire 60 seconds of image data at a sampling rate of 30 images per second, for an image data set of approximately 1800 images.

At 108, the method performs one or more quality checks to evaluate the quality of the obtained signals for WBC count determination. In examples, these quality checks include motion detection, magnetic field strength, and light intensity. A signal from an RGB motion sensor is evaluated to determine if there are motion artifacts in the acquired images. Motion may be evaluated on a pixel-by-pixel variation or by movement of a reference point. In still other examples, motion may be determined by the application of a supervised learning model modeling motion artifacts and/or noisy versus stable signal features. In examples, there may be two kinds of motion evaluations performed. A local motion parameter looks at the amount of motion permitted between a few successive frames while a global motion parameter defines an amount of motion permitted from the starting frame to the final frame. A magnetometer, which may be incorporated in the circuitry of the smartphone 22, is positioned near the camera 18 operates to measure the magnetic field in the vicinity of the patient's finger. A measurement from the magnetometer is checked to confirm that the magnetic field from the magnet 12 to which the finger and the electronics are subjected is within an operational range. In examples, the magnetic field has a strength within a range of 1.0 mT-2 T. In one example reduced to practice, the magnetic field had a strength of 1100 μT. Threshold values may be set for the evaluation of local motion and for global motion. Too strong of a magnetic field may interfere with data collection and analysis while too weak of a magnetic field will not achieve the RBC regulation as intended. Prior to insertion of a patient's finger, the system may capture one or more images of the light from the light source 14, exemplarily filtered by filter 16. This forms a baseline check as to the initial wavelength and intensity of the light to which the finger is exposed.

If motion artifacts, magnetic field, or light intensity checks do not meet predefined limits, then a notification may be presented indicating the type of quality check that was not passed and the need to re-collect video data. The process is repeated until the signal from the RGB motion sensor indicates that the acquired images are free of motion artifacts and any other quality checks are passed. Recognizing that ability to limit finger movement may vary among patients (e.g. children or arthritic patients), in examples, the motion artifact threshold may be an adjustable parameter. This may be done so with the understanding that increased motion artifact allowance may lower the confidence interval of the resulting WBC count. Upon completion of the data acquisition sub-process, the method 100 proceeds to the data analysis sub-process.

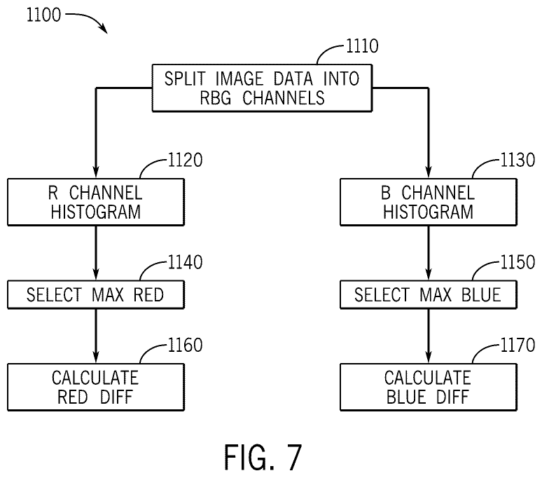

The presently disclosed data analysis sub-process uses a pixel-based analysis of each of the image frame from the image data set. At 110, the image data is processed on a frame (e.g. image) by frame basis to extract image attributes from each of the about 1800 images. FIG. 7 is a flow chart of an example of the method 1100 of image attribute extraction as provided herein. Through experimentation and investigation, the inventors have discovered that following a pixel-based analysis, the following four image features may be extracted and advantageously used in non-invasively determining WBC count. However, it is recognized that this determination may be non-limiting and that other image attributes may be used in addition or instead of the following features while remaining within the scope of the present disclosure.

| Feature Name | Definition |
|---|---|
| MaxRed | the maximum pixel intensity value of red channel in a |
| | frame |
| MaxBlue | the maximum pixel intensity value of red channel in a |
| | frame |
| RedDiff | Difference between the red channel maximum histogram |
| | (MaxRed) values of in two successive frames |
| BlueDiff | Difference between the blue channel maximum histogram |
| | (MaxBlue) values in two successive frames |

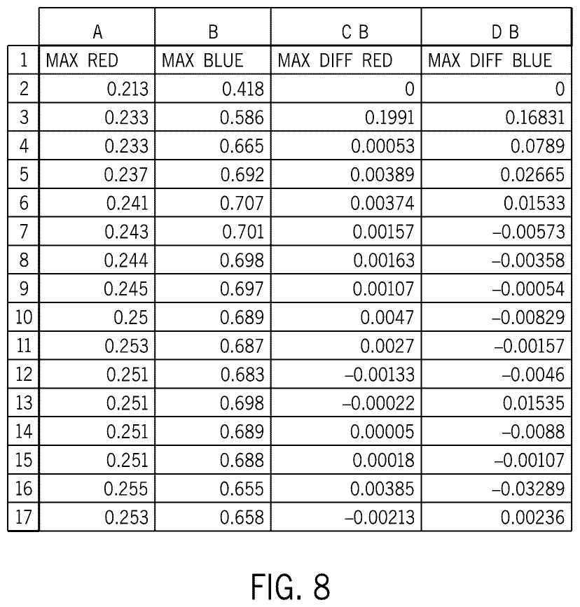

The individual images (frames) of the image set (video) are sequentially numbered and the above features are extracted from each of the images (frames). FIG. 8 is a portion of an example of the extracted features as presented in a tabular form organized by sequential image number. Each image was divided into Red, Green, and Blue channels and evaluated on a pixel-by-pixel basis, for example to produce a histogram of pixel values in each of the Red, Green, and Blue channels. An RGB (color) histogram is a representation of the distribution of colors in an image. Features are extracted on a frame-by-frame basis. The MaxRed feature is the maximum pixel intensity in the red channel of the frame. The MaxBlue feature is the maximum pixel intensity in the blue channel of the frame. These MaxRed and MaxBlue values are stored and selected for each image. The RedDiff and Blue Diff values are comparative values. The RedDiff value is the difference between the MaxRed value in the current frame and the MaxRed value in the immediately preceding image (frame). The BlueDiff value is the difference between the MaxBlue value in the current frame and the MaxBlue value in the immediate preceding frame.

In further description, for each frame of the fingertip video, the cv2.VideoCapture( ) Python script can be used to read the input fingertip video and assign it to a variable named “capture” (capture=cv2.VideoCapture(video_inputFile). At 1110 of the method 1100, the video data frames are splint into RGB channels. The capture.read( ) Python script is used to access each frame and cv2.split( ) used on each frame to obtain blue, green, and red channel values. This splits the video data into RGB channels and calculates each channel's pixel values. Next, a red histogram is calculated at 1120 for the red channel and a blue histogram is calculated at 1130 for the blue channel of each frame. This is exemplarily calculated using cv2.calcHist( ) The histograms may further be normalized based upon the number of pixels per frame. At 1140 the MaxRed value is selected from the red histogram and at 1150 the MaxBlue value is selected from the blue histogram. At 1160 the RedDiff value is calculated by subtracting the MaxRed value of the previous frame from the MaxRed value of the current frame. At 1170 the BlueDiff value is calculated by subtracting the MaxBlue value of the previous frame from the MaxBlue value of the current frame.

Returning to FIG. 6, at 112 unsupervised machine learning is applied to the extracted features to categorize the image data. In an example, K-means clustering technique is applied to the extracted features to group the frames (images) based upon the data contained therein. Identifying that there are four “bands” of fluorescence data, in e.g. the Red channel, coinciding with varying blood compositions, the inventors sought to use four clusters, for example, starting with randomly positioned centroids, and clustered across all four extracted features. These clusters correspond to RBC, RBC & Platelets, Platelets, and WBC. The K-means clustering technique assigns each data point to the closest centroid to form a K cluster. The centroid positions are optimized to cluster the data to minimize intra-cluster dissimilarity and maximize inter-cluster dissimilarity.

In a further example of this analysis, the K-means clustering is applied to determine the cluster-assignment threshold value for each of the clusters. For example, the following Python script: (kmMaxRed=KMeans(n_clusters=4,init=“random”, n_init=2, max_iter=10000).fit(dfMaxRed)) may be used to start the K-means clustering with k=4. The MaxRed values are read using the variable dfMaxRed. This script uses a csv file generated from the input video file (dfMaxRed=pd.read_csv(inputFile, usecols=[“maxRed”])). Additionally, the following scripts may be used in the above analysis: KMeans( ).fit(dfMaxRed); centersMaxRed=kmMaxRed.cluster_centers_; Counter (kmMaxRed.labels_).

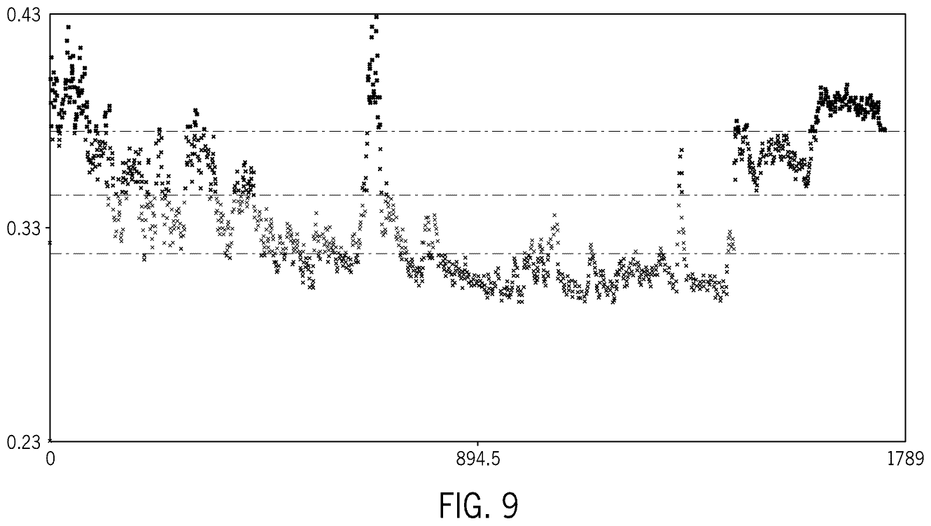

Once classified with these clusters, at 114, a rate of WBC is calculated. The MaxRed feature is further evaluated on an frame by frame basis. An example plot of the classified MaxRed data by image is presented at FIG. 9. Knowing that WBC return the least intensity in the red channel compared to RBC (high) and platelets (medium), the lowest-intensity cluster is classified as data associated with WBC. Further features are extracted from this classification. Temporally, each MaxRed intensity drop into the WBC classification is counted and noted. For each drop, a number of frames and the duration of each drop is counted. The table below provides an example of these output values from the data presented in FIG. 9.

| | | MaxRed | | |
|---|---|---|---|---|
| Cluster | Cluster # | Threshold | Frame Count | % of Total |
| WBC | 0 | 0.306 | 685 | 38% |
| PLT | 1 | 0.3279 | 415 | 23% |
| RBC + PLT | 2 | 0.3609 | 418 | 23% |
| RBC | 3 | 0.3888 | 272 | 15% |
| Total | N/A | 0.3365 (avg) | 1790 | 100% |

From the analysis above in the example data of FIG. 9, 38% of the blood flow during the 60 seconds of which the image data was acquired was attributed to WBC. While the output of this analysis produces a WBC rate of 38%, volumetric quantification of blood flow in analyzed anatomy is needed to calculate the WBC count. WBC count is typically expressed as a number of WBC per unit of blood volume (either WBC #/Liter or WBC #/μL). This requires a determination of the amount of blood flowing through the analyzed anatomy over a fixed amount of time. With the above calculation of the rate distribution of blood composition, in the analyzed anatomy, the present system thus requires a determination of the amount of blood through the analyzed anatomy over the same time.

Cardiac output (CO) is the volume of blood pumped by the heart per minute. Cardiac output is the product of heart rate (HR) and stroke volume (SV) and is measured in liters per minute. Three manners of determining CO at 116 are described herein, although persons of ordinary skill in the art will also recognize other manners of determining CO, suitable for use with examples of this method.

In a first example, an estimate of CO, or an estimated CO range is made based upon patient physiological information including, but not limited to sex, weight, and height. This basic patient physiological data may be collected during an initial set-up for WBC count monitoring. CO estimates or estimate ranges based upon basic patient physiological data are described by St Pierre, S. R., Peirlinck, M., & Kuhl, E. (2022). Sex Matters: A Comprehensive Comparison of Female and Male Hearts. Frontiers in physiology, 13, 831179; Patel N, Durland J, Makaryus A N. Physiology, Cardiac Index. [Updated 2022 Sep. 26]. In: StatPearls [Internet]. Treasure Island (FL): StatPearls Publishing; 2023 January; and Proctor D N, Beck K C, Shen P H, Eickhoff T J, Halliwill J R, Joyner M J. Influence of age and gender on cardiac output-VO2 relationships during submaximal cycle ergometry. J Appl Physiol 1998; 84:599-605, each of which are incorporated by reference herein in their entireties. From these or other references, an estimated CO range may be determined from the basic patient physiological data and stored as a static reference value for use as described herein.

A localized temperature-based approach may be used in estimating localized blood flow. A ring with a heat source chip and a temperature sensor is placed on the finger to be imaged in the WBC count analysis. The heat source chip operates to heat the finger and generates heat diffusion between the chip and the temperature sensor. The temperature sensor is designed to measure the temperature difference in order to quantify blood flow velocity through the finger. Since the blood flow is the main medium of heat diffusion in bodies, part of the heat energy in the tissue will be taken away by the flowing blood. Therefore, the blood flow velocity can be acquired via this relationship with the temperature difference. Relying on these data, a fitting curve can be established and consequently and equation made to related blood flow velocity with the temperature measured at the temperature sensor.

A further example as will be described in further detail herein estimates CO from the acquired image data. As described in US2021/0007648, the acquired video data can be used for Photoplethysmography (PPG) analysis, from which CO can be estimated. A PPG system requires a light source to illuminate the tissue and a photodetector to capture the variation of the light intensity. The intensity variations are observed due to the systole and diastole effects of the heart. There are two general types of PPG: transmission and reflection. Transmission PPG is used on thin anatomy (e.g. finger, earlobe), while reflection PPG can be used across a wider range of patient anatomy. The charged-coupled device (CCD) camera of a typical modern smartphone can capture signals in a NIR range suitable for PPG acquisition. The patient's finger covers the smartphone camera lens without additional pressure. The volumetric changes in arterial blood are captured by calculating the changes in light absorption in the images of the video data. Since the video captures a continuous sequence of frames, a continuous PPG signal can be calculated from the captured frames and used for real-time volumetric quantification of blood flow. Features can be extracted from this signal for either reflection or transmission PPG. PPG features include, but are not limited to, systolic and diastolic peak, normalized PPG rise time, pulse transit time (PTT), pulse shape, and amplitude.

PPG can measure blood volume changes in the micro-vascular bed of tissue using optical properties of blood. The PPG signal is non-stationary and quasi-periodic where Fourier analysis applies to stationary periodic signals. Fourier Series analysis can be applied to PPG signals on a cycle-by-cycle basis. To remove high-frequency noise of a PPG signal, the data is filtered using smoothing filters for examples Savitzky-Golay (SG) smoothing filter, Butterworth filter, Gaussian filter. Later, the cycle-by-cycle Fourier Series (CFS) analysis can be carried out. This method may reduce the measurement error of the PPG signal by a factor of 10 or more. Butterworth filtering flattens the frequency response of a signal in the passband.

As described in US2021/0007648, the image data can be processed to for PPG in the following manner. Each image (frame) is subdivided into blocks and a time series signal is generated for each block across all of the frames. A band pass filter is applied to each time series signal. In one example, a band pass filter of 0.66 Hz-8.33 Hz may be used, with a minimum cut-off value selected to discard the signal fluctuations due to breathing or other motion artifacts. The processed signal data is evaluated for three PPG cycles where each cycle includes a systolic peak and a diastolic peak. If three continuous PPG cycles are not found, then select at least one cycle which has a systolic peak and a diastolic peak, replicate the selected cycle three times, and combined them to create a three-cycle PPG signal. Features are extracted from this three-cycle PPG signal. The features may include the ratio of AC and DC components, systolic peak, diastolic peak, a slope of each peak, a relative timestamp value of the peak, dicrotic notch height, ratio, and augmented ratio among systolic, diastolic, and dicrotic notch, systolic and diastolic rising slope, and inflection point area ratio. Finally, the extracted features may be averaged.

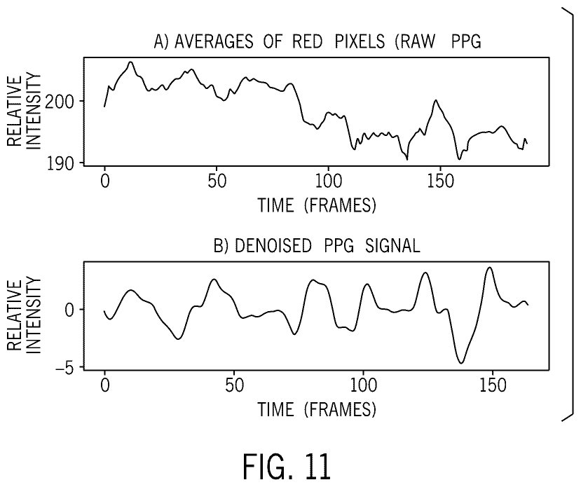

In a further modification of the PPG analysis, the time series signals generated from each block across all of the frames may be smoothed using a Butterworth filter. Considering each frame as a sample point, the value of the Nyquist frequency in the Butterworth filter is half of the sampling rate r. In the frequency domain, FFT magnitudes are generated, FFT noise reduction is performed, and frequency peaks are identified as stored in a peak indices list. The frequencies of the peak indices list are multiplied by 60 to obtain a BPM (beat per minute) value, used for further processing of the PPG signal. In an example the following Python scripts may be used to carry out this analysis: scipy.signal.butter( ) scipy.signal.filtfilt( ) np.fft.fft, and np.fft.ifft( ) The script scipy.signal.find_peaks( ) is applied to the smooth signals. FIG. 11 presents comparative graphs of a raw PPG signal calculated from the red channel values of the image data and a denoised PPG signal therefrom after the processing described above.

A PPG cycle is extracted from the times series PPG data by maintaining a threshold of 0.8 BPM to 1.2 BPM. A PPG cycle is analogous to a cardiac cycle that repeats in a PPG signal. The peak finding functions thus can be used to extract the PPG cycles from the signal. From this, the features of maxima indices, minima indices, peak indices, start of PPG indices, and end of PPG indices. In this example, rather than extracting or generating three or three consecutive PPG signals, all PPG signals meeting threshold criteria to be deemed to be high quality or best quality signals are selected. This selection may be based upon a comparison of the features extracted above. In an example the PPG signals with the least deviation from the BPM threshold are selected.

An inflection point area (IPA) is calculated from the selected PPG signals. The IPA being the ratio of the area after the inflection point to the whole area under the PPG curve in the time domain. This may be calculated using trapezoidal integration. A normalized harmonic area (NHA) is calculated in the frequency domain features like frequency and magnitude of n-th harmonics. The Python script np.fft.fftfreq( ) may be used to convert the time varying signal to the frequency domain magnitudes. The base and following frequency are calculated by multiplying the magnitude of the n-th maxima index with the sampling rate, the ratio of the number of frames to video duration. The base and the following magnitude values were determined by dividing the magnitude of the n-th maxima index with the PPG cycles length. Inflection harmonic area ratio (IHAR) is calculated as the ratio of IPA to NHA. Cardiac output can be calculated from IHAR.

According to wave reflection theory, arterial blook pulse can be divided into two waves: a first wave produced by the heart pumping and a second wave produced by pulse wave reflection. Therefore, the inflection point area (IPA) ratio, the area ratio of the second and first peak in the PPG wave is mainly influenced by the strength of the pulse wave reflection. The pulse wave reflection alters both the time and frequency domains of the PPG signal. Previous research demonstrated a strong correlation between the normalized harmonic area (NHA), derived from frequency domain analysis, and pulse wave reflection as well as systolic and diastolic blood pressure. Thus, IHAR, calculated by dividing NHA by IPA may be used as an indictor of cardiac output. In an example, CO may be calculated from IHAR using the equation CO=α·IHAR+β, wherein the coefficients α and β may be empirically derived.

The WBC count can be calculated at 118 from the ratio of WBC to Total Blood Cells (WBC rate) calculated at 114 and the cardiac output during the same duration as the analyzed video data (e.g. 60s). The WBC count can be reported as WBC #/μL.

Lastly, at 120, the WBC count is interpreted to a WBC level by applying the WBC count to clinically relevant WBC count thresholds. In an example, the following ranges are used to provide a WBC level. However, other populations and demographics may exhibit difference ranges for each level. Across all populations, normal is generally considered between 4000-5000/μL and 10,000-11,000/μL.

| Level | Range |
|---|---|
| High | WBC count > 11,000/μL |
| Normal | 4000/μL < WBC count < 11,000/μL |
| Low | 1800/μL < WBC count < 4,000/μL |
| Severe (e.g. neutropenia) | WBC count < 1800/μL |

The computer 20 operates to present the determined WBC level on the graphical display 38 to inform the patient and clinician.

In an exemplary summary of the above, the following parameters are used in the equations:
Video duration (t), in seconds
Number of frames in video (f)
Number of frames in histogram lowest intensity-cluster (β)
Ratio of WBC to total blood cells (r)
Blood flow amount (α)
WBC count, (C)
   Ratio of WBC to total blood cells (r) is calculated from the histogram-based matrix data. It is calculated by dividing the number of frames in the lowest intensity cluster (β) with the sum of elements in all clusters, exemplarily the number of frames (f) in the video.

$$r = \frac{β}{f} \qquad ( 1 )$$

The blood flow amount (α) in the analyzed anatomy is calculated by normalizing the blood flow rate, the volume flow rate (VFR) to μl/s units.

$$α = \frac{V F R * t}{30} * 50 \qquad ( 2 )$$

The resulting count of the WBC (C) is the ratio of the WBC to total blood cells (r) to the blood flow amount (α).

$$C = \frac{r}{α} \qquad ( 3 )$$

The systems and methods for extra-clinical, non-invasive WBC count as described herein can provide advantages for a variety of patients. In some examples, patients may be able to eliminate or reduce in-clinic phlebotomy and laboratory blood work. In an extra-clinical setting, a patient may be able to provide the WBC count in telemedicine communications, giving a clinician an additional piece of information in making a recommendation for an in-person, urgent, or emergency care visit. In a case of a patient undergoing chemotherapy, frequent monitoring of WBCs can prompt an intervention in response to a sudden drop in WBCs.

In examples wherein the same patient receives repeated measurements of WBC count, the models used herein may be further receive an initial calibration using a WBC count from a laboratory CBC test and image data collected contemporaneously to the blood draw for the laboratory test. This calibration can help to personalize the model to the individual patient, providing additional accuracy in WBC count determination. If the system is used for consistent, ongoing WBC count monitoring, the periodic laboratory CBC test WBC count information can be added to the analysis, maintaining calibration to the patient over time.

Implementations as described herein may leverage the computing power and data collection capabilities of a common smart phone device. Recognizing a variety of smart phone cameras and capabilities, it will be recognized that various implementations may require adjustments to account for specific operating features of the smartphone used. Such accommodations may include adjusting for processing power, computational resources, camera resolution, IR and magneto sensors. Additionally, the position of the camera varies between phone makes and models, which may result in physical adjustments to the system for alignment with camera position.

While a system with local processing has been described herein, it will be recognized that in an alternative example, some or all of the data processing may be performed using remote and/or cloud computing resources. In such an example, the patient may be registered through a secure platform to handle the transmission of the patient's medical data. The patient registration record may include, but not limited to, their basic physiological information and a record of previous WBC count determinations. The patient records the exemplary one minute video of image data and submits the video along with their registration credentials to upload the data to the remote and/or cloud based system for applying the analysis and data processing as described above. The calculated WBC count is sent back to the smartphone 22 to be presented on the display 38. Additionally, trends or comparative results based upon the determined WBC level may be presented to the patient through the display.

In the above description, certain terms have been used for brevity, clarity, and understanding. No unnecessary limitations are to be inferred therefrom beyond the requirement of the prior art because such terms are used for descriptive purposes and are intended to be broadly construed. The different systems and method steps described herein may be used alone or in combination with other systems and methods. It is to be expected that various equivalents, alternatives, and modifications are possible within the scope of the appended claims.

The functional block diagrams, operational sequences, and flow diagrams provided in the Figures are representative of exemplary architectures, environments, and methodologies for performing novel aspects of the disclosure. While, for purposes of simplicity of explanation, the methodologies included herein may be in the form of a functional diagram, operational sequence, or flow diagram, and may be described as a series of acts, it is to be understood and appreciated that the methodologies are not limited by the order of acts, as some acts may, in accordance therewith, occur in a different order and/or concurrently with other acts from that shown and described herein. For example, those skilled in the art will understand and appreciate that a methodology can alternatively be represented as a series of interrelated states or events, such as in a state diagram. Moreover, not all acts illustrated in a methodology may be required for a novel implementation.

This written description uses examples to disclose the invention, including the best mode, and also to enable any person skilled in the art to make and use the invention. The patentable scope of the invention is defined by the claims and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A system for non-invasive measurement of white blood cell (WBC) count, the system comprising:
a camera configured to acquire a plurality of images;
a light source;
a magnet positioned relative to the camera; and
a processor configured to receive the plurality of images, extract at least one image attribute from each image of the plurality of images, apply a K-means clustering technique to the extracted image attributes to classify the plurality of images, calculate a WBC rate from the classified images, and use a cardiac output with the WBC rate to calculate the WBC count.

2. The system of claim 1, further comprising a graphical display, wherein the controller is configured to determine a WBC level from the WBC count and visually present the WBC level on the graphical display.

3. The system of claim 1, wherein the light source is configured to emit light about 430 nm wavelength.

4. The system of claim 3, further comprising a polarizing band pass filter configured with a pass band centered on 430 nm wavelength.

5. The system of claim 1, wherein the system is configured for a user to insert a finger along a first direction to position the finger relative to the camera and the magnet is positioned to produce a magnetic field generally parallel to the first direction.

6. The system of claim 1, wherein the image attributes extracted from each image in of the plurality of images comprise: a peak value of light absorbed in a red channel and a difference between two successive peak values of light absorbed in the red channel.

7. The system of claim 6, wherein the image attributes extracted from each image in the plurality of images comprises: a peak value of light absorbed in a blue channel and a difference between two successive peak values of light absorbed in the blue channel.

8. The system of claim 1, further comprising a magnetometer positioned proximate to the camera, wherein the processor is configured to measure a magnetic field proximate to the camera with the magnetometer and compare the measured magnetic field to a threshold value before receiving the plurality of images.

9. The system of claim 1, wherein the processor is further configured to calculate the cardiac output from the plurality of images.

10. The system of claim 1, wherein the extracted at least one image attribute is a maximum pixel intensity value and the K-means clustering classifies the at least one extracted image attribute into a plurality of K-clusters, wherein the processor is configured to calculate WBC rate using a K-cluster of the plurality of K-clusters selected as having a lowest maximum pixel intensity threshold value.

11. The system of claim 10, wherein the maximum pixel intensity value is a maximum pixel intensity value in a red channel of each image.

12. The system of claim 11, wherein the processor is configured to calculate WBC rate as a count of the images in the selected K-cluster divided by a count of the number of images classified.

13. The system of claim 10, wherein the plurality of K-clusters comprises four clusters, and wherein a first cluster having a highest maximum pixel intensity threshold value, a second cluster having a second highest maximum pixel intensity threshold value, a third cluster having a third highest maximum pixel intensity threshold value, and a fourth cluster having the lowest maximum pixel intensity threshold value, and wherein the fourth cluster is the selected cluster.

14. A method of non-invasive measurement of white blood cell (WBC) count, the method comprising:
illuminating a target with light centered on a 430 nm wavelength;
subjecting the target to a static magnetic field;
acquiring a plurality of images of the target;
extracting image attributes from each image of the plurality of images;
classifying the images of the plurality of images using a K-means classifying algorithm to define classification boundaries to a plurality of K-clusters;
calculating a WBC rate from the classified images; and
calculating the WBC count from the WBC rate and a cardiac output.

15. The method of claim 14, wherein the WBC rate is calculated from red channel image data from the classified images.

16. The method of claim 14, further comprising:
obtaining the cardiac output from a lookup table based upon patient demographic information; or
calculating cardiac output from the plurality of images of the target, and optionally:
obtaining a photoplethysmography (PPG) signal from the plurality of images;
calculating an inflection point area (IPA) from the PPG signal;
obtaining a normalized harmonic area (NHA) from the PPG signal;
calculating an inflection harmonic area ratio (IHAR) between the IPA and the NHA; and
calculating cardiac output from the IHAR.

17. The method of claim 14, further comprising applying clinical standards to the calculated WBC count to determine a WBC level.

18. The method of claim 14, further comprising:
measuring a magnetic field with a magnetometer positioned proximate the camera; and
confirming a strength of the static magnetic field before acquiring the plurality of images of the target.

19. The method of claim 14, further comprising:
measuring at least one of a magnetic field, a light intensity, and a patient motion; and
comparing the at least one of the measured magnetic field, light intensity, and patient motion against at least one respective predetermined threshold before acquiring the plurality of images of the target.

20. (canceled)
